(19) United States
(12) Patent Application Publication
(10) Pub. No.: US 2023/0389847 A1
(43) Pub. Date: Dec. 7, 2023

(54) ELECTRODE SYSTEM FOR RUBBER EAR TIPS WITH CONDUCTIVITY FROM N-DOPED SILICONE OR CONDUCTIVE FILAMENTS IN MIXTURE FOR ELECTROENCEPHALOGRAPHY

(71) Applicant: Niura Corporation, Kew Gardens, NY (US)

(72) Inventors: Ryan Farzam Ahmed, Kew Gardens, NY (US); Shahriar Huda, Jamaica, NY (US); Authoy Das, Jamaica, NY (US); Steffani Rajapaksha, Bayonne, NJ (US); Melani Shrestha, Elmhurst, NY (US); Alisha Karim, Queens, NY (US); Chloe Kan, Oakland Gardens, NY (US); Arun Chhugani, Bethlehem, PA (US); Chengling Zheng, Flushing, NY (US); Genevive Chang, New York City, NY (US); Dang Nguyen, Wellington, FL (US); Pari Patel, Hagerstown, MD (US); Jinnat Mahmood, Jamaica, NY (US); Katherine Xiao, Watkinsville, GA (US); Lesley Osasogie Iyamu, Mount Vernon, NY (US); Mohammad Rahim, Jamaica, NY (US); Nana Adom Opare-Addo, Bronx, NY (US); Dhriti Rathod, Chambersburg, PA (US); Sakef Uddin, Jamaica, NY (US); Samiha Nasrin, Bronx, NY (US); Saqib Khan, Elmont, NY (US); Saifa Sowa, Hollis, NY (US); Tanvi Jammula, Ashburn, VA (US); Veda Kolli, Ballwin, MO (US); Wajiha Moin, Brooklyn, NY (US); William Yu, Bayside, NY (US); Deven Rozario, Flushing, NY (US)

(73) Assignee: Niura Corporation, (omitted)

(21) Appl. No.: 18/454,063

(22) Filed: Aug. 23, 2023

(51) Int. Cl. A61B 5/263; A61B 5/374; A61B 5/00; H04R 1/10

(52) U.S. Cl. CPC A61B 5/263; A61B 5/374; A61B 5/6817; H04R 1/1016

(57) ABSTRACT

The present disclosure involves the integration of conductive filaments or n-doped silicon with a rubber ear-tip. This combination allows for the detection of brain oscillation waves in EEG-enabled earbud systems. Previous implementations of ear tip electrodes involve multiple tiny electrodes embedded into the ear tip. However, unlike conventional designs, the presented solution utilizes the entire ear tip for conductivity, making a significant advancement in EEG technology in regards to data capturing. Other embodiments of the invention include partitioning the ear tip itself to make an electrode array for multiple reference points during detection of brain waves. These conductive ear tips enable accurate neural biometric detection. Incorporating magnets, the ear tips seamlessly attach to earbuds, enhancing convenience.

### PRIOR PUBLICATION DATA

US/2022/63/373,331

### RELATED U.S. APPLICATION DATA

CONVERSION OF PROVISIONAL (UTILITY) PATENT APPLICATION No. 63/373,331 FILED ON Aug. 24, 2022

RELATED NONPROVISIONAL (UTILITY) patent application Ser. No. 18/452,456 FILED ON Aug. 19, 2023

### FIELD OF CLASSIFICATION SEARCH

REFERENCES CITED:

| | |
|---|---|
| 10,835,145 B1 11/2020 Prevoir | A61B 5/0478 |
| 11,344,256 B2 05/2022 Kirszenblat | A61B 5/0408 |
| 2020/0129102 A1 04/2020 Singh | A61B 5/14551 |
| 2023/0240611 A1 12/2022 Khaleghimeybodi | A61B 5/6817 |
| 11,571,563 B2 02/2023 Hanson | A6IN 1/0496 |

### FIELD OF THE INVENTION

N-doped or conductive silicone ear tips are used as flexible electrodes for electroencephalography (EEG) to allow detection of neural biometrics. The field of this invention encompasses the intersection of various domains including bioelectronics, biometric analysis, neuroscience, audio engineering, medical devices, and health screening. Additionally, the invention deals with the measurement and analysis of brain activity through EEG, contributing to brain research and the development of new medical devices for diagnosing and monitoring neurological conditions. Integrating biological signals with engineering principles to develop an innovative solution for a medical application also falls within the realm of audio and biomedical engineering.

### BACKGROUND

Description of the Problem: Existing methods for integrating EEG (electroencephalography) technology into earbuds have certain limitations that hinder their effectiveness. Previous solutions embed metal electrodes within silicone ear tips, failing to fully utilize the ear tip's surface for conductivity, leading to less reliable and suboptimal EEG data capture. Traditional earbuds also lack advanced electrode materials like n-doped silicone or conductive filaments, preventing them from effectively detecting a wide range of brainwave frequencies. Moreover, the absence of magnetic implementation in current designs hampers easy attachment and alignment of the ear tips with the earbud apparatus. As a result, the demand for an improved electrode system that maximizes conductivity, enhances EEG data accuracy, and ensures user comfort within the context of EEG-enabled earbuds remains unmet. This innovation seeks to address these challenges and offer a comprehensive solution that harmonizes advanced materials, enhanced conductivity, and optimal electrode positioning, thereby advancing EEG technology and enabling more accurate brainwave measurements through wearable earbuds in various settings.

Description of the Related Art: Recent filings by BOSE, a company that focuses on manufacturing and selling audio equipment, described an invention of ear tips being used as electrodes. However, this invention only encompasses the implementation of metallic electrodes ingrained in the silicon ear tip; it does not permit the entire ear tip to be conductive so that the entire apparatus serves as an electrode. Patent US 2020/0129102 A1 describes a concept for wearable devices with integrated circuitry. This invention incorporates an elastomeric material and electronic components. The manufacturing process involves embedding electrical pathways into the elastomeric material and integrating a skin-contacting sensor. However, users of this device may potentially experience discomfort during use due to the sensor's functional dependability, as the device may be prone to damage. Additionally, inconsistent readings may result from the device's motion during usage. Another similar filing is patent U.S. Ser. No. 11/344,256B2 which utilizes an electrode on the eartip and another on the external portion of the earpiece. Other patents such as U.S. Pat. No. 8,428,287B2 use rubber elastomers to discharge electrostatic discharge and protect users. This usage of rubber elastomers utilizes internal circuitry to resist rather than conduct electricity. This does not hold any biometric capturing capabilities and would not be effective in addressing the current problem. Current earbuds are very similar with minimal variation between brands, however, they do not contain an n-doped silicone or conductive rubber mixture, which is crucial for efficient electrical conductivity. Furthermore, they do not have a magnetic implementation to allow for easy placement of the ear tips onto the earbud apparatus.

Objective of the Invention: The invention implements an enhanced electrode system that harmonizes the design and functionality via the integration of rubber ear tips with electrical conductivity. The implementation of n-doped silicone and conductive filaments achieves the desired enhancement of electrical conductivity for the invention. The conductivity boost introduced by n-doped silicone or conductive filaments creates an efficient pathway for electrical signals to traverse, thereby minimizing distortions and signal degradation. The refined electrode system ensures optimal contact between the electrodes and the skin, minimizing impedance, and attaining an elevated level of signal quality notably enhancing the accuracy and dependability of EEG data capture. Incorporating materials characterized by strong conductivity contributes to generating precise outcomes in EEG data.

Brief Summary of the Invention: The invention descriptive herein pertains to an ear tip composed of conductive filaments or n-doped silicon electrodes. With this implementation, the conductive ear tips are capable of detecting brain oscillation waves from various regions of the brain. This technical design differs from traditional methods that involve multiple metal electrodes embedded within a normal rubber ear tip. Instead, the present invention is entirely made out of conductive filaments, reducing the need to insert additional metal electrodes. A pivotal feature of the present disclosure is its ability to detect an extensive range of brainwave frequencies, encompassing alpha, beta, theta, delta, and gamma waves. These frequencies captured by the present invention can be further analyzed for comprehensive biometric analysis. Furthermore, the design considers potential partitioning of the conductive ear tip, allowing for a grid formation. Other embodiments include the integration of conductive filaments and n-doped silicon electrodes on the entirety of the earbud apparatus. Such a configuration is anticipated to augment the surface area contact within the ear, enhancing the granularity of EEG data capture.

### Technical Terminology and Concepts

Electroencephalogram (EEG): A screening and diagnostic method employed to measure and record electrical activity generated by the brain. This is done using electrodes placed on the scalp, which capture fluctuations in voltage that result from ionic current flows within the neurons of the brain.

Biocompatibility: Biocompatibility refers to the ability of a material to interact with biological systems without causing harm or adverse reactions. n-doped silicon, mentioned in the patent, is described as biocompatible, indicating that it can be safely used in contact with the skin.

Electrodes: Conductive devices or mediums designed to detect electrical signals. In the context of the present invention, these are integrated into earbuds to facilitate the non-invasive capture of EEG data.

Electrode System: A complex arrangement of electrodes and other conductive materials that achieve electrical contact between a biological surface and device with the purpose of capturing or measuring electrical signals.

EEG-Integrated Earbuds: An integration of EEG technology into earbuds. This involves the incorporation of electrodes within the earbuds' structure, enabling the continuous capture of electrical signals from the brain which is then formulated into EEG data. This happens all while performing the primary function of audio delivery.

N-doped Silicone: A nitrogen-integrated, silicone-based material with enhanced conductive properties, biocompatibility, flexibility, and durability. The material serves as a conductive substrate which enables greater skin-electrode contact, thereby enhancing signal quality during EEG data capture.

Conductive Filament: Thin materials composed of polymers and metals that typically possess high levels of electrical conductivity. They are employed to create low-resistance pathways for the transmission of electrical signals while maintaining flexibility and adaptability.

Bioimpedance: The measure of the opposition to the flow of an alternating electric current within biological tissues, often used to assess parameters like body composition and hydration.

Neural Biometrics: The use of unique patterns or characteristics derived from an individual's brain activity or neural responses as a means of identifying and authenticating that individual.

Ground Electrode: The main role of the ground is to shield the delicate biopotential signals of interest from any interference caused by power line noise.

Amplification: Amplification involves increasing the strength or magnitude of electrical signals. In EEG systems, amplification is used to boost the weak electrical signals generated by the brain so that they can be accurately recorded and analyzed.

Soft Metals: Soft metals are malleable and have relatively low melting points. These metals include tin, lead, and alloys like solder. The mixture of soft metals within the conductive ear tip material aids in establishing reliable electrical contact between the electrode and the skin.

Brain-Activity Interface: The brain-activity interface refers to the interaction between the brain's electrical signals and external devices. In this context, the conductive filaments or n-doped silicon electrodes in the ear tips act as an interface to capture and transmit brainwave activity to external devices for analysis.

Delta, Theta, Alpha, Beta, and Gamma Waves: Different types of brainwave frequencies that represent varying states of brain activity. Delta waves are associated with deep sleep, theta waves with relaxation, alpha waves with wakeful relaxation, beta waves with active thought and concentration, and gamma waves with high-level cognitive processing.

### SUMMARY

According to a report released by the United Nations, neurological disorders are alarmingly prevalent around the globe, affecting up to one billion individuals and resulting in up to 6.8 million deaths annually. This high incidence of brain-related issues is exacerbated heavily by a lack of early detection methods. While traditional EEG solutions capture reliable and comprehensive data, they are heavily limited in their scope. Electroencephalography caps are cumbersome and limited to use in clinics and are impractical in surgical contexts. More invasive solutions, while offering a wider range of usage, come with their own set of risks, including but not limited to intracranial hemorrhaging. All of these solutions are also costly to patients, creating financial barriers that hinder access to essential neurological monitoring and diagnosis. Accessibility to a cost-effective and portable alternative has the potential to democratize EEG monitoring, ensuring that individuals from diverse socioeconomic backgrounds can benefit from timely and accurate neurological assessment, ultimately leading to improved health outcomes.

The present innovation combines conductive electrode technology within an ear tip, creating a screening tool capable of measuring neural activity. Prioritizing user comfort and experience, the portable EEG signal capture mode with in-ear EEG systems offers profound utility and benefits to enhance the understanding of cognitive processes and improve early detection. The EEG device, which is noninvasive and user-friendly, can be utilized in various scenarios. This enables brain monitoring to be available in a range of environments, such as surgical rooms to outdoor settings. By seamlessly integrating portable EEG screening in a passive and continuous manner, this technology fosters familiarity along with practicality among users. The technology enhances audio and musical connectivity through shared listening experiences.

The invention involves an electrode system to enhance the accuracy of EEG data collection. Conductive filaments are embedded into the silicone electrodes, playing a crucial role in detecting and interpreting alpha, beta, theta, and gamma brainwaves. The silicone electrodes are integrated into rubber ear tips, consisting of soft metals within the mold mixture to optimize the conduction of electricity and signal recognition. These neural signals are translated into user-friendly insights through a brain-activity interface, providing users with an understanding of the user's brainwave activity.

### BRIEF DESCRIPTIONS

In the following portion relating to the detailed description, the embodiments of the present disclosure will be explained more in detail with reference to the example figures of the proposed invention shown in the drawings, which:

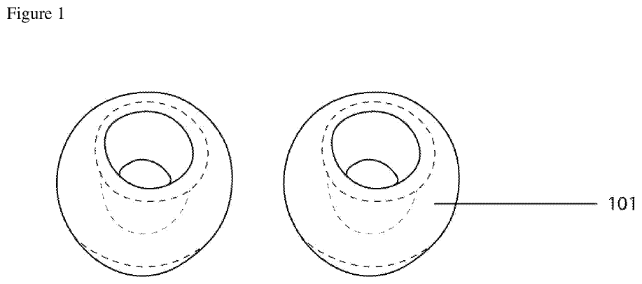

FIG. 1 depicts a front-view perspective of two n-doped silicon electrodes or flexible conductive material required in an EEG earbud system.

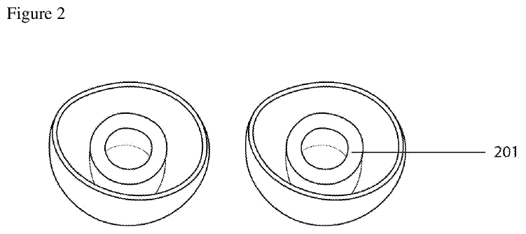

FIG. 2 portrays an aerial view of the n-doped silicon electrode or flexible conductive material placed in an upside-down orientation.

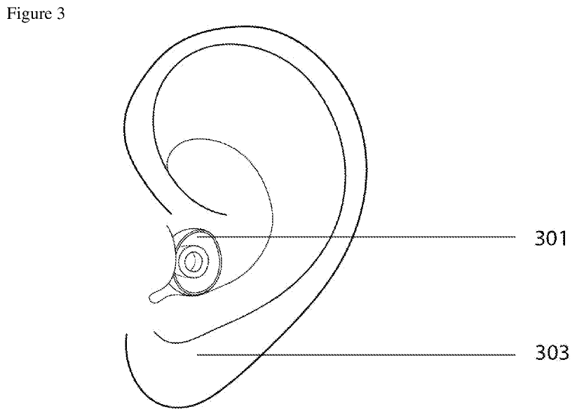

FIG. 3 depicts an upright orientation of n-doped silicon electrode or flexible conductive material placed in the ear.

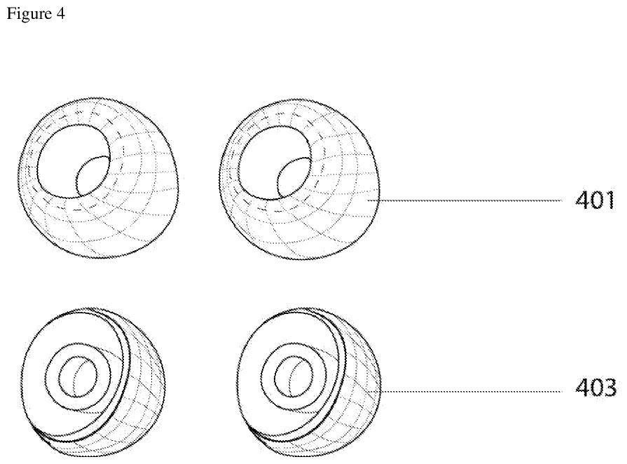

FIG. 4 is an aerial perspective of another embodiment that illustrates a potential with multiple partitions and an electrode array within the silicon electrode itself.

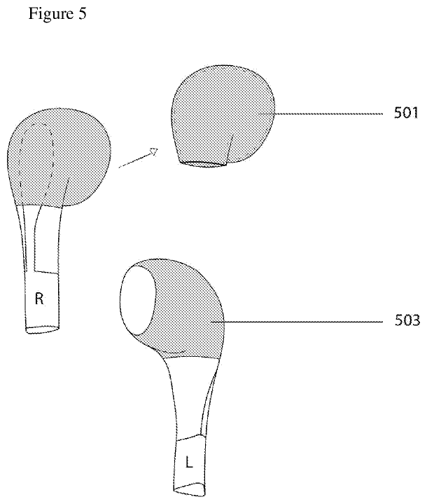

FIG. 5 displays a side-view perspective of another embodiment where the entire earbud-body apparatus is composed of robust conductive filaments or n-doped silicon electrodes.

101 depicts the frontal orientation of a set of two eartips. These eartips are composed of conductive n-doped silicon or flexible material such as rubber or silicone and a mixture of metallic materials. Rather than serving solely as passive audio conduits, these ear tips transform into fully integrated electrodes. This transformation enables them to capture not only auditory sensations but also intricate brainwave signals for biometric analysis and EEG. 101 varies in dimensions of ear tip depth, height, and diameter in order to fit users' preferences.

### DETAILED DESCRIPTION

The present invention discloses conductive rubber ear tips capable of recording electroencephalographic data while prioritizing user experience.

101 depicts the frontal orientation of a set of two eartips. These eartips are composed of conductive n-doped silicon or flexible material such as rubber or silicone and a mixture of metallic materials. Rather than serving solely as passive audio conduits, these ear tips transform into fully integrated electrodes. This transformation enables them to capture not only auditory sensations but also brainwave signals for biometric analysis and EEG. 101 varies in dimensions of ear tip depth, height, and diameter in order to fit users' preferences. These silicone ear tips will be connected to the rest of the earbud by way of 201, which depicts the rear view of two ear tips. Here, the n-doped silicon electrode or flexible conductive material is placed in an upside-down orientation. The back end of the ear tip, 201, contains magnets that connect directly with the brain's electrical signals, ensuring that electrical activity can be captured and transmitted to external devices.

301 demonstrates this ear tip in use, portraying an upright orientation of an ear tip placed within the preauricular pit of the human ear. The user's ear 303 assumes the role of the recipient of auditory experiences and monitors their brainwave activity as transmitted to external devices. The ear tips will be made to securely fit inside the external auditory canal, 301, directing sound waves into the recesses of the ear canal. This will improve the audio quality of the earbuds, as well as the brain wave readings. Additionally, the secure fit will serve the purpose of making sure there is adequate surface contact between the eartip and the user, 303. Without adequate contact, the signal cannot accurately be detected and the signal that is detected can be filled with noise and interference. With this secure fit, the user can be assured that the gathered data is correct and reliable.

401 provides greater insight into the orientation of the electrodes on the ear tip. Since the ear tip can be comprised of either n-doped silicone or conductive filaments, the entire ear tip itself can serve as an electrode. The grid orientation on the surface of the eartip 401 (front view) and 403 (back view) visualize the placement of each electrode. Every electrode will be placed within a grid box, ensuring maximum contact and more accurate EEG data readings. Surface contact between the electrode and the user's ear, 303, will allow the electrode to detect brain activity and have the data sent to any device programmed to receive the information.

While this style of traditional silicone eartips is relatively common, this invention can be adapted to other forms of eartips or earbud coverings. FIG. 5 displays an earbud design without a silicone eartip, where the earbud body can be inserted directly into the user's ear canal. As seen in FIG. 5, this system can be adapted to any shape or size to have perfect fitment and be compatible with any and all earbuds on the market and in the future market. Similarly to 401, this iteration is made out of a conductive material, which includes but is not limited to n-doping silicone or the use of conductive filaments. This conductive material surrounds the entire top body or cover of the earbud, 503, allowing the earbud cover to receive electrical signals such as brain activity.

The apparatus is distinctly designed to incorporate a hard conductive rubber cover, 501, enveloping the earbud's exterior. This conductive rubber cover, 501, offers an improved user experience in comparison to traditional plastic materials, as it has been thoughtfully selected for its ergonomic properties to provide enhanced comfort during prolonged usage. In addition to prioritizing user comfort, the hard conductive rubber cover also houses strategically positioned EEG electrodes, thereby enabling the continuous acquisition of EEG data during the earbud's operation. The utilization of conductive rubber for hosting EEG electrodes ensures the direct and efficient transduction of neural signals to electrical signals, which can be processed and analyzed by external devices.

The embodiment also features a hard conductive rubber cover, 503, applied to the main body of the earbud. This second conductive rubber cover, 503, is designed to harmonize with the first cover, 501, both in terms of material and function. Its presence on the earbud body serves not only to maintain a consistent aesthetic but also to provide additional EEG electrode placements for comprehensive brainwave activity detection.

1. An electrode system for rubber and/or silicone ear tips with conductivity, comprising:
a. Conductive filaments or n-doped silicon electrode(s) integrated into an ear tip structure
b. Said conductive filaments or n-doped silicon electrode(s) configured to detect brain oscillation waves from various regions of the brain;
c. Said ear tip structure entirely composed of conductive filaments or n-doped silicon electrode(s) without the inclusion of traditional metal electrodes.

2. The electrode system of claim 1, wherein the conductive filaments are configured, but not exclusive to, in a grid formation within the ear tip structure.

3. The electrode system of claim 1, wherein the conductive ear tip detects a range of brainwave frequencies, including alpha, beta, theta, delta, and gamma waves.

4. The electrode system of claim 1, wherein the ear tip structure is further integrated into an earbud apparatus, compromising:
a. Conductive filaments or n-doped silicon electrode(s) covering the entirety of the earbud apparatus;
b. Enhanced surface area contact within the ear, enabling improved granularity of EEG data capture.

5. The electrode system of claim 1, wherein the conductive ear tip is able to be used for delivering sound to the ear playing music, acoustic, etc.

6. The electrode system of claim 1, wherein the ear tips are electrically connected to earbuds or other audio devices to provide a method of EEG data capture.

7. A method for improving EEG data capture using an electrode system for rubber ear tips with enhanced electrical conductivity, comprising:
a. Eartip structure from rubber or silicon material being manipulated with n-doped silicon or conductive filaments into the structure to enhance electrical conductivity;
b. Inserting the ear tip into the ear to establish electrode-skin contact;
c. Facilitating efficient transmission of brain oscillation waves through the enhanced electrical conductivity, resulting in minimized signal degradation and distortion;
d. Capturing EEG data with elevated accuracy and dependability due to the improved electrode-skin contact and reduced impedance.

8. The method of claim 7, wherein the enhanced electrical conductivity of the ear tip contributes to the reduction of noise and interference during EEG data capture, leading to improved data fidelity and interpretability.
